(19) United States
(12) Patent Application Publication
(10) Pub. No.: US 2004/0069612 A1
(43) Pub. Date: Apr. 15, 2004

(54) Substrate processing apparatus and substrate processing method

(76) Inventors: Yukihiko Nakata, Yokohama-shi (JP); Tetsuya Okamoto, Yokohama-shi (JP); Kazufumi Azuma, Yokohama-shi (JP); Masashi Goto, Yokohama-shi (JP)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/673,173

(22) Filed: Sep. 30, 2003

(30) Foreign Application Priority Data
Sep. 30, 2002 (JP) 2002-285876

(51) Int. Cl. C07C 1/00

(52) U.S. Cl. 204/157.15; 204/193

(57) ABSTRACT

A substrate processing apparatus includes a light source, a plurality of light transmitting windows, and a reaction chamber, in which a substrate is placed. And a surface of the substrate, which opposes the light transmitting windows is processed by using a reaction which occurs when the light from the light source is irradiated into the reaction chamber through the light transmitting windows. This substrate processing apparatus includes a driving mechanism which moves the substrate relative to the light transmitting windows in a direction parallel to the surface. The width of each of the light transmitting windows in the direction in which the substrate moves relative to the light transmitting windows is smaller than the length of the substrate in the moving direction.

### CROSS-REFERENCE TO RELATED APPLICATIONS

This application is based upon and claims the benefit of priority from the prior Japanese Patent Application No. 2002-285876, filed Sep. 30, 2002, the entire contents of which are incorporated herein by reference.

### BACKGROUND OF THE INVENTION

1. Field of the Invention

The present invention relates to an apparatus for performing reactions, such as photo-oxidation, photo-CVD, photo-ashing, photo-cleaning, photo-etching, and photo-epitaxy, which occur when a reduced-pressure gas is irradiated with light from a light source. For example, the present invention relates to a substrate processing apparatus for use in the fabrication process of semiconductor devices.

2. Description of the Related Art

For example, a FET (Field Effect Transistor) having a MOS (Metal Oxide Semiconductor) structure or a polysilicon TFT (Thin Film transistor) has a semiconductor layer and/or insulating film. A plasma is sometimes used in the formation and oxidation of this semiconductor layer and/or insulating film. However, if the film formation or oxidation is performed using a plasma, it is difficult to completely avoid ion damage caused by this plasma.

As methods of avoiding this ion damage, photo-oxidation, photo-CVD (Chemical Vapor Deposition), photo-ashing, photo-cleaning, photo-etching, photo-epitaxy, and the like are known.

Conventionally, the following methods are disclosed as examples of photo-oxidation in “Y. Nakata, T. Okamoto, T. Hamda, T. Itoga, and Y. Ishii: Proceedings of Int. Conf. on Rapid Thermal Processing for Future Semiconductor Devices (2001)”, “Y. Nakata, T. Okamoto, T. Hamda, T. Itoga, and Y. Ishii: Proceedings of Int. Workshop on Gate Insulator 2002 (2001)”, “Y. Nakata, T. Okamoto, T. Hamda, T. Itoga, and Y. Ishii: Proceedings of Asia Display/IDW′01 p. 375 (2001)”, and “Y. Nakata, T. Itoga, and Y. Ishii: 2001 Spring 48th Applied Physics Related Joint Lecture Meeting (Tokyo)”.

An ambient containing oxygen gas is irradiated with light of a xenon (Xe) excimer lamp, and the surface of a semiconductor is oxidized by the formed active oxygen atoms. In this manner, a first insulating film is formed on the semiconductor surface. After that, a second insulating film is formed by plasma CVD by using a gas mixture of TEOS (Tetra Ethyl Ortho Silicate) and O₂, or a gas mixture of SiH₄ and N₂O.

The method of producing the active oxygen atoms by using light as described above causes no ion damage, so good interfaces can be formed. However, the conventional substrate processing apparatus for performing this photo-oxidation has the following problem.

To explain this problem of the conventional substrate processing apparatus, FIG. 12 shows a schematic side view of the conventional substrate processing apparatus for forming an insulating film by an oxidation reaction using light.

In FIG. 12, reference numeral 301 denotes xenon excimer lamps as light sources; 302, a lamp house as a light source unit; 304, a light transmitting window made of synthetic quartz; 305, a vacuum reaction chamber (also called a vacuum chamber: to be referred to as a reaction chamber hereinafter); 306, a substrate having a semiconductor surface; 307, a substrate holder on which the substrate 306 is placed; 308, a gas inlet from which oxygen gas is supplied; and 310, a vacuum evacuate port from which air in the reaction chamber 305 is evacuated. Nitrogen gas (N₂ gas) is sealed in the lamp house 302 to obtain a substantially atmospheric pressure. The area of the light transmitting window 304 is made larger than that of the upper surface of the substrate 306. Accordingly, the entire upper surface of the substrate 306 is irradiated with light from the xenon excimer lamps 301.

An insulating film is formed on the substrate 306 by using the conventional substrate processing apparatus shown in FIG. 12 as follows. First, the substrate 306 is loaded into the reaction chamber 305 and held on the substrate holder 307. After air in the reaction chamber 305 is once evacuated, an oxygen gas is introduced in the reaction chamber 305. The substrate 306 is irradiated, through the light transmitting window 304, with 172 nm-wavelength light emitted from the xenon excimer lamps 301. Consequently, the semiconductor surface of the substrate 306 is oxidized to form an insulating film on this surface.

When the short-wavelength light from the xenon excimer lamps 301 exposed to the air, it decomposes oxygen molecules in the air into active oxygen atoms, and is absorbed by an air layer of a few millimeters thick. To avoid this light absorption, therefore, the lamp house 302 formed on the synthetic quartz light transmitting window 304 is usually filled with nitrogen gas which does not absorb light having a wavelength of 172 nm at substantially the atmospheric pressure.

To suppress impurities in an insulating film to be formed, the reaction chamber 305 in which the substrate 306 is placed to be oxidized is once evacuated, and then oxygen gas is supplied into the reaction chamber 305 to keep a desired pressure. After that, the reaction chamber 305 is irradiated with the light through the light transmitting window 304. This light decomposes oxygen molecules to active oxygen atoms, thereby oxidizing the semiconductor surface of the substrate 306.

In this method, however, a gas pressure difference between a substantially atmospheric pressure and a pressure near a vacuum, i.e., a pressure of about 1 kg/cm² (9.80665×10⁴ Pa) apply on the light transmitting window 304. Therefore, the thickness of the light transmitting window 304 must be so set as to withstand this pressure.

Table 1 below shows the size of a synthetic quartz plate, the thickness of each synthetic quartz plate necessary to withstand the gas pressure difference described above, and the transmittance of light having a wavelength of 172 nm with respect to each synthetic quartz plate. As shown in Table 1, when the light transmitting window 304 is a circular window having a diameter of 300 mm or a square window of 250 mm side, the thickness of the light transmitting window 304 must be about 30 mm.

TABLE 1
172 nm-wavelength light

| The size of | Diameter | Diameter | | |
|---|---|---|---|---|
| the synthetic | of 6″ | of | 250 mm | 300 mm |
| quartz plate | (15.24 cm) | 300 mm | square | square |
| The thickness | 4.3 mm | 3.0 mm | 30.6 mm | 36.8 mm |
| of the | | | | |
| synthetic | | | | |
| quartz plate | | | | |
| Transmittance | 45% | 30% | 30% | 25.60% |

FIG. 11 shows the relationship between the light wavelength and the light transmittance with respect to the synthetic quartz plates (thickness=1, 10, and 30 mm).

As shown in FIG. 11, the transmittance of light having a wavelength of 172 nm with respect to the synthetic quartz plate abruptly lowers when the thickness of this synthetic quartz plate is increased. When the thickness of the synthetic quartz plate is 30 mm, the light transmittance is about 30%. That is, when the thickness of the synthetic quartz plate is 30 mm, an effectively usable portion of the light reduces to ⅓ or less. This extremely largely lowers the oxidation rate. In a substrate processing apparatus for a large square substrate of about 1 m square, the synthetic quartz thickness becomes impractically thick.

### BRIEF SUMMARY OF THE INVENTION

A substrate processing apparatus according to an aspect of the present invention is a substrate processing apparatus which comprises a light source, at least one light transmitting window which transmits light from the light source, and a reaction chamber capable of being evacuated, and in which a substrate to be processed is placed in the evacuated reaction chamber so as to oppose the light transmitting window with a spacing between them, and at least a surface to be processed of the substrate, which opposes the light transmitting window is processed by using a reaction which occurs when light from the light source through the light transmitting window is irradiated into the reaction chamber, comprising a driving mechanism which moves the substrate relative to the light transmitting window, wherein the width of the light transmitting window in the direction in which the substrate moves relative to the light transmitting window is set to be smaller than the length of the substrate in the moving direction.

This invention comprises the driving mechanism which moves the substrate relative to the light transmitting window. Therefore, the width of the light transmitting window in the moving direction can be made smaller than the length of the substrate in the moving direction.

When the substrate processing apparatus has a plurality of light transmitting windows, these light transmitting windows can be juxtaposed in a first direction in which the substrate to be processed is moved, or can be juxtaposed in the first direction and a second direction different from the first direction.

When the light transmitting windows are to be juxtaposed in the first direction and the second direction different from the first direction, these light transmitting windows are preferably arranged into a check pattern.

The driving mechanism is preferably a mechanism which swings the substrate with respect to the light transmitting windows, or a mechanism which moves the substrate in one direction with respect to the light transmitting windows.

When the mechanism which swings the substrate with respect to the light transmitting windows is to be used as the driving mechanism, the light transmitting windows are preferably juxtaposed in the moving direction such that the widths of the light transmitting windows in the swinging direction are constant, and intervals between adjacent light transmitting windows in the swinging direction are constant, and the stroke of the swing by the driving mechanism is preferably set to be larger than a repeating interval which is the sum of the width in the swinging direction of the light transmitting window and the width in the swinging direction of a beam formed between adjacent light transmitting windows.

When the substrate processing apparatus has a plurality of light transmitting windows, these light transmitting windows may also be juxtaposed in the moving direction such that intervals between adjacent light transmitting windows in the moving direction are not uniform.

When the driving mechanism is the mechanism which moves the substrate in one direction with respect to the light transmitting windows, the length of the reaction chamber in the moving direction is favorably twice the length of the substrate in the moving direction or more.

Preferably, the reaction chamber has a gate valve, at least one sub-reaction chamber different from the reaction chamber is placed adjacent to the reaction chamber via the gate valve, and the driving mechanism moves the substrate in one way from the reaction chamber to the sub-reaction chamber over the gate valve.

The light source is favorably a low-pressure mercury lamp, rare gas excimer lamp, or xenon excimer lamp.

A substrate processing method according to another aspect of the present invention comprises steps of placing a substrate to be processed in an evacuated reaction chamber of a substrate processing apparatus comprising a light source, at least one light transmitting window which transmits light from the light source, and the reaction chamber capable of being evacuated, such that the substrate opposes the light transmitting window with a spacing between them, irradiating into the reaction chamber with the light from the light source through the light transmitting window, while moving the substrate relative to the light transmitting window such that the substrate is parallel to the light transmitting window, and processing at least a surface to be processed of the substrate, which opposes the light transmitting window, by a reaction which occurs when the interior of the reaction chamber is irradiated with the light from the light source.

In this invention, while the substrate to be processed is moved relative to the light transmitting window, the light from the light source through the light transmitting window is irradiated into the reaction chamber. Therefore, the substrate to be processed can be processed even if the width of the light transmitting window in the moving direction is smaller than the length of the substrate in the moving direction.

Preferably, this invention further comprises steps of preparing a substrate to be processed having a surface to be processed which is at least partially made of a semiconductor, and forming an ambient containing at least oxygen gas in the reaction chamber, and the step of processing at least the surface to be processed of the substrate by the reaction which occurs when the light from the light source is irradiated into the reaction chamber comprises a step of oxidizing the surface to be processed by using active oxygen atoms formed by the reaction which occurs when the light from the light source is irradiated into the reaction chamber, thereby forming an insulating film on the substrate.

Alternatively, the method can further comprise a step of forming, in the reaction chamber, an ambient of a gas of a compound having an atom which belongs to group 14 (C, Si, Ge, Sn, and Pb) of the periodic table or a gas mixture containing the gas, an ambient of a gas mixture containing a gas of a compound having an atom which belongs to group 13 (B, Al, Ga, In, and Tl) of the periodic table and a gas of a compound having an atom which belongs to group 15 (N, P, As, Sb, and Bi) of the periodic table, an ambient of a gas mixture containing a gas of a compound having an atom which belongs to group 12 (Zn, Cd, and Hg) of the periodic table and a gas of a compound having an atom which belongs to group 16 (O, S, Se, Te, and Po) of the periodic table, or an ambient of a gas containing at least a silicon compound gas, and the step of processing at least the surface to be processed of the substrate by the reaction which occurs when the light from the light source is irradiated into the reaction chamber can comprise a step of forming a semiconductor film on the substrate by the reaction which occurs when light from the light source is irradiated into the reaction chamber.

Photo-oxidation, photo-CVD, photo-ashing, photo-cleaning, photo-etching, or photo-epitaxy can be used as the reaction which occurs when light from the light source is irradiated into the reaction chamber the through at least one light transmitting window.

Also, at least two of photo-oxidation, photo-CVD, photo-ashing, photo-cleaning, photo-etching, and photo-epitaxy can be continuously performed without breaking a vacuum.

Additional objects and advantages of the invention will be set forth in the description which follows, and in part will be obvious from the description, or may be learned by practice of the invention. The objects and advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate presently preferred embodiments of the invention and, together with the general description given above and the detailed description of the preferred embodiments given below, serve to explain the principles of the invention.

FIG. 1 is schematic view showing a substrate processing apparatus according to the first embodiment of the present invention;

FIG. 2 is schematic view showing a substrate processing apparatus according to the second embodiment of the present invention;

FIG. 3 is a top view showing light transmitting windows without a lump house of the substrate processing apparatus according to the second embodiment of the present invention;

FIG. 4A is a schematic view showing the state in which photo-oxidation is performed by a substrate processing apparatus according to the third embodiment of the present invention;

FIG. 4B is a schematic view showing the state in which plasma CVD is performed by the substrate processing apparatus according to the third embodiment of the present invention;

FIG. 5 is a top view showing parts of light transmitting windows of a substrate processing apparatus according to the fourth embodiment of the present invention;

FIG. 6 is a cross section view showing parts of light transmitting windows of a substrate processing apparatus according to the fifth embodiment of the present invention;

FIG. 7 is a top view showing parts of light transmitting windows of a substrate processing apparatus according to the sixth embodiment of the present invention;

FIG. 8 is a chart for joining FIG. 8A, FIG. 8B and FIG. 8C together.

FIGS. 8A, 8B and 8C is a process flow chart for fabricating a polysilicon thin-film transistor by using the substrate processing apparatus according to the sixth embodiment of the present invention;

FIGS. 9A to 9E are cross sectional views each showing a process of fabricating the polysilicon thin-film transistor by using the substrate processing apparatus according to the sixth embodiment of the present invention;

FIG. 10 is a schematic view showing the substrate processing apparatus according to the sixth embodiment of the present invention;

FIG. 11 is a graph showing the dependence of the transmittance of a synthetic quartz plate upon the wavelength; and

FIG. 12 is a schematic view showing the conventional substrate processing apparatus.

### DETAILED DESCRIPTION OF THE INVENTION

Embodiments of the present invention will be described in detail below with reference to the accompanying drawing. In the drawing explained below, the same reference numerals denote parts having the same functions, and a repetitive explanation thereof will be omitted.

### First Embodiment

The first embodiment of the present invention will be described below with reference to FIG. 1. The first embodiment will be explained by taking, as an example, a substrate processing apparatus for forming an insulating film on a substrate to be processed by photo-oxidation. As a substrate 6 to be processed, it is possible to use a substrate having a surface 6a to be processed which is at least partially made of a semiconductor, e.g. a single-crystal Si substrate. As the substrate 6, it is also possible to use, e.g., a substrate obtained by forming a semiconductor layer on one surface of a glass substrate.

The substrate processing apparatus comprises a plurality of xenon excimer lamps 1 as light sources, a lamp house 2 housing the lamps 1, at least one, e.g., six light transmitting windows 4a to 4f for transmitting light from the lamps 1, a vacuum reaction chamber (to be referred to as a reaction chamber hereinafter) 5, a substrate 6 to be processed, a substrate holder 7 on which the substrate 6 is placed, a driving mechanism 34, and the like. The reaction chamber 5 can be evacuated. Reference numeral 10 in FIG. 1 denotes a vacuum exhaust port from which air in the reaction chamber 5 is exhausted. Also, reference numeral 8 in FIG. 1 denotes a gas inlet from which a gas contained in a gas cylinder (not shown) is supplied into the reaction chamber 5. When an insulating film is to be formed on the substrate 6 by photo-oxidation as in the first embodiment, a gas containing oxygen atoms, e.g., oxygen (O₂) gas is supplied from the gas inlet 8 as indicated by an arrow X in FIG. 1.

The plurality of (in the first embodiment, six) xenon excimer lamps 1 for emitting light having a wavelength of 172 nm are, for example, have a linear shape (like round rods). In the lamp house 2, the lamps 1 are arranged parallel to each other to extend in the direction perpendicular to the paper of FIG. 1. In the lamp house 2, a gas such as nitrogen gas (N₂ gas) which does not easily absorb the light from the xenon excimer lamps 1 is sealed to set a substantially atmospheric pressure.

Between the lamp house 2 and a surface 6a to be processed of the substrate 6, the light transmitting windows 4a to 4f for transmitting the light from the xenon excimer lamps 1 are made by, e.g., synthetic quartz. However, the material of the light transmitting windows 4a to 4f is not limited to synthetic quartz; the light transmitting windows 4a to 4f need only be made by an optically transparent material. The light transmitting windows 4a to 4f are juxtaposed in a first direction (the horizontal direction in FIG. 1) so as to correspond to the linear xenon excimer lamps 1 juxtaposed to each other.

In FIG. 1, reference numeral 32 denotes a support member as a supporting means for supporting the light transmitting windows 4a to 4f; and 15, beams for supporting the six light transmitting windows 4a to 4f. The support member 32 has an opening 32a. The plurality of, e.g., five beams 15 are extended across the opening 32a at predetermined intervals. In this way, the opening 32a is divided into six small openings. The support member 32 has a receiving portion 33 which horizontally extends to the edge of the opening 32a. Each of the beams 15 has a pair of receiving portions 15a which horizontally extend from the two side edges.

Each of the light transmitting windows 4a to 4f has a pair of extending edges 31 which horizontally extend from the two side edges. The extending edges 31 of the light transmitting windows 4a to 4f are engaged with the receiving portions 15a and 33. In addition, O-rings made of rubber (not shown) are inserted between the extending edges 31 and the receiving portions 15a and 33 to keep the airtightness between them, thereby fixing the light transmitting windows 4a to 4f to the beams 15 and support member 32. In this manner, the light transmitting windows 4a to 4f close the individual small openings. Note that the support member 32 and beams 15 may also be integrated.

The moving mechanism 34 moves the substrate 6 relative to the light transmitting windows 4a to 4f in a direction parallel to the surface 6a to be processed. The moving mechanism 34 can be a mechanism which moves the substrate 6 or a mechanism which moves the light transmitting windows 4a to 4f. The direction in which the moving mechanism 34 moves the substrate 6 relative to the light transmitting windows 4a to 4f can be any arbitrary direction provided that the direction is parallel to the surface 6a to be processed with respect to the light transmitting windows 4a to 4f, and that the surface 6a is evenly processed by recovering a decrease in illuminance under the beams 15. The driving mechanism 34 of the first embodiment swings the substrate holder 7 with respect to the light transmitting windows 4a to 4f, thereby swinging the substrate 6 placed on the substrate holder 7 with respect to the light transmitting windows 4a to 4f. The swinging direction of the substrate 6 and substrate holder 7 is a direction indicated by an arrow B1 in FIG. 1. In the first embodiment, the moving direction, i.e., the sliding direction of the substrate 6 (indicated by the arrow B1 in FIG. 1) is the same as the first direction along which the light transmitting windows 4a to 4f are juxtaposed. A stroke S of the swinging motion of the substrate 6 by the moving mechanism 34 is 35 mm. The moving mechanism 34 can be the existing moving mechanism such as a moving mechanism which comprises an actuator and a control circuit for controlling the operation of the actuator.

A width W_{W} in the above-mentioned moving direction of the light transmitting windows 4a to 4f is smaller than a length W_{B} of the substrate 6 to be processed. In the first embodiment, the width W_{W} of the light transmitting windows 4a to 4f is 25 mm, a width D of the beams 15 is 5 mm, a thickness T of the light transmitting windows 4a to 4f is 5 mm, and the transmittance to 172-nm light of the light transmitting windows 4a to 4f is 65%. In the conventional substrate processing apparatus, the light transmitting window is, e.g., a circular window having a diameter of 6 inches. The transmittance of this 6-inch circular light transmitting window is 45%. Accordingly, the light transmittance of the light transmitting windows 4a to 4f is made higher than that of the conventional 6-inch circular light transmitting window.

In addition, in the first embodiment, the sum (in the first embodiment, 30 mm) of the width D (5 mm) of the beam 15 and the width W_{W} (25 mm) of the light transmitting window is constant, i.e., the distance between the adjacent light transmitting windows in the moving direction (the distance between the centers of the adjacent light transmitting windows) is constant. The sum of the width D and width W_{W} will be referred to as a repeating interval C hereinafter.

A substrate processing method will be explained below.

First, a circular P-type single-crystal Si wafer having a (100) surface, resistivity of 10 to 15 Ωcm and a diameter of 6 inches is prepared as the substrate 6 to be processed. The substrate 6 is cleaned and transferred to the substrate holder 7 in the evacuated reaction chamber 5 via a loading chamber (not shown). The substrate 6 is set on the substrate holder 7 heated to 300° C. by a heater (not shown). In this state, the (100) surface of the substrate 6 is the upper surface. This (100) surface of the substrate 6 is the surface 6a to be processed. A distance D2 between the light transmitting windows 4a to 4f and substrate 6 is 5 mm.

Next, oxygen gas is supplied from the gas inlet 8 at a flow rate of 50 sccm. While the internal pressure of the reaction chamber 5 is held at 70 Pa, the substrate holder 7 is swung as described previously. When light is emitted from the xenon excimer lamps 1 having a wavelength of 172 nm in this state, the oxygen gas is directly and efficiently decomposed to produce highly active oxygen atoms. In this state, the oxygen gas partial pressure is about 70 Pa. This active oxygen atoms oxidizes the (100) surface of the substrate 6, forming an oxide film (SiO₂ film) on the substrate 6. This oxide film will be referred to as a first insulating film hereinafter.

In the first embodiment using the reaction chamber 5 and xenon excimer lamps 1, active oxygen atoms O(¹D) can be efficiently formed directly from oxygen as indicated by reaction formula (1) below. The active oxygen atoms O(¹D) oxidizes the surface of a semiconductor layer (the (100) surface of the substrate 6). When the xenon excimer lamps 1 are used as described above, ozone does not participate in the reaction.

Xenon excimer lamp

O₂+hν→O(³P)+O(¹D)(wavelength 172 nm) (1)

O(³P): oxygen atom in ³P-level excited state

O(¹D): oxygen atom in ¹D-level excited state

h: Planck's constant

ν: frequency of light

It is also possible to use a low-pressure mercury lamp as a light source. The wavelength of light emitted by a low-pressure mercury lamp has two peaks, i.e., 185 and 254 nm. When a low-pressure mercury lamp is used, therefore, as indicated by reaction formula (2) below, 185 nm light produces ozone from oxygen, and this ozone forms active oxygen atoms O(¹D) by 254 nm light. That is, this reaction is a two-stage reaction.

Low-pressure mercury lamp

O₂+O(³p)+M→O₃+M (wavelength 185 nm) (2)

O₃+hν→O(¹D)+O₂ (wavelength 254 nm) (3)

M: oxygen compound gas except O₂, O(³P), and O₃

The reaction caused by the xenon excimer lamp 1 is a one-stage reaction. Therefore, compared to a low-pressure mercury lamp, the active oxygen atoms O(¹D) can be formed very efficiently, so the oxidation rate is high. Note that the reaction indicated by reaction formula (1) occurs when light having a wavelength of 175 nm or less is used.

Oxidation has two modes: one is “reaction-rate control” by which the oxidation rate is determined by the rate of the reaction between silicon and oxygen; and the other is “diffusion-rate control” by which the oxidation rate is determined by the rate at which the oxidation species diffuses in an oxide film and reaches the interface between a silicon oxide film (SiO₂ film) and silicon (Si). When the temperature of a single-crystal Si wafer increased, the rate of the reaction between silicon and oxygen also rises, but particularly the rate at which the oxidation species diffuses in an oxide film increases. Therefore, the oxidation rate increases when the temperature of the single-crystal Si wafer is raised. When the influence on the substrate processing apparatus and single-crystal Si wafer (substrate 6 to be processed) is taken into consideration, the semiconductor temperature during photo-oxidation is preferably 100° C. to 500° C., and more preferably, 200° C. to 350° C. In the first embodiment, the semiconductor temperature is 300° C.

In the substrate processing apparatus of the first embodiment, an oxide film (SiO₂ film) about 4.3 nm was formed by photo-oxidation in 90 min. The uniformity of the oxide film was ±70% without any swing, and was improved to ±7% when the substrate was swung. Also, the uniformity of the oxide film thickness was improved by making the stroke S (35 mm) of the swing of the substrate 6 larger than the repeating interval C (30 mm) of the light transmitting windows 4a to 4f. In addition, the irradiating light intensity in the first embodiment was 11 mW/cm² at the position of the substrate 6. The throughput was improved by using the xenon excimer lamps 1 as light sources.

The capacitance-voltage characteristic was measured by using an electric capacitance measurement sample formed by stacking first and second insulating films on the substrate 6 to be processed.

The second insulating film can be formed by, e.g., the following method. To eliminate a tunnel current to allow easy measurement of the semiconductor-insulating film interface state density, a second insulating film (SiO₂ film) about 94 nm thick is formed on the substrate 6 on which the first insulating film is formed as described above. The second insulating film can be formed by a CVD apparatus, different from the substrate processing apparatus of the first embodiment, by using TEOS gas and O₂ gas. After that, an aluminum film is formed by sputtering on the second insulating film (SiO₂ film) formed over the (100) surface of the substrate 6. Then, a large number of circular dot patterns 0.8 nm in diameter made of an aluminum film are formed by photolithography.

The capacitance-voltage characteristic was measured by using the thus formed electric capacitance measurement sample. As a consequence, the interface fixed charge density was 1×10¹¹ cm⁻², i.e., equivalent to that of a thermal oxide film (an SiO₂ film formed by thermally oxidizing the (100) surface of an Si substrate).

As described above, the first embodiment is a substrate processing apparatus comprising the xenon excimer lamps 1 as light sources, the light transmitting windows 4a to 4f as at least one light transmitting window which transmits light from the xenon excimer lamps 1, and the reaction chamber 5 which can be evacuated. The substrate 6 to be processed is placed in the evacuated reaction chamber 5 so as to oppose the light transmitting windows 4a to 4f with a spacing between them. The light from the xenon excimer lamps 1 is irradiated into the reaction chamber 5 through the light transmitting windows 4a to 4f. By using the reaction caused by this irradiation, at least the surface 6a to be processed, i.e., the (100) surface of the substrate 6, which opposes the light transmitting windows 4a to 4f is processed. The apparatus further comprises the driving mechanism 34 for moving the substrate 6 relative to the light transmitting windows 4a to 4f in the direction parallel to the surface 6a to be processed. In addition, the width W_{W} of the light transmitting windows 4a to 4f in the direction along which the substrate 6 and light transmitting windows 4a to 4f move relative to each other is set to be smaller than the length W_{B} of the substrate 6 in this moving direction.

In the first embodiment, therefore, the light transmitting windows 4a to 4f can be made smaller than the conventional ones, so their thickness can also be made smaller than that of the conventional ones accordingly. This makes it possible to suppress absorption (loss) of light from the light sources 1 by the light transmitting windows 4a to 4f, and increase the oxidation rate. It is also possible to decrease the weights of the materials forming the windows 4a to 4f and beams 15 regardless of the size of the substrate 6 to be processed. Consequently, the substrate processing apparatus can be manufactured inexpensively.

The light transmitting windows 4a to 4f are juxtaposed in the first direction, e.g., the direction (indicated by the arrow B1 in FIG. 1) parallel to the moving direction described above. Furthermore, the driving mechanism 34 swings the substrate 6 with respect to the light transmitting windows 4a to 4f. The stroke S of this swing is set to be larger than the repeating interval C of the light transmitting windows 4a to 4f. By this arrangement, the substrate 6 to be processed can be evenly processed.

Also, the substrate processing method of the first embodiment comprises the step of preparing the substrate 6 to be processed having the surface 6a to be processed which is at least partially made of a semiconductor, the step of placing the substrate 6 in the evacuated reaction chamber 5 of the substrate processing apparatus which comprises the lamps 1, the light transmitting windows 4a to 4f for transmitting light from the lamps 1, and the reaction chamber 5 which can be evacuated, such that the substrate 6 opposes the light transmitting windows 4a to 4f with a spacing between them, the step of forming an ambient containing at least oxygen gas in the reaction chamber 5, the step of irradiating into the reaction chamber 5 with light from the lamps 1 through the light transmitting windows 4a to 4f while moving the substrate 6 relative to the light transmitting windows 4a to 4f in a direction parallel to the surface 6a to be processed, and the step of oxidizing the semiconductor surface as the surface 6a of the substrate 6 by using the active oxygen atoms formed by the reaction which occurs when the light from the lamps 1 is irradiated into the reaction chamber 5, thereby forming an insulating film on the substrate 6.

By this method, while the substrate 6 to be processed is moved relative to the light transmitting windows 4a to 4f in the direction parallel to the surface 6a to be processed, the interior of the reaction chamber 5 is irradiated with the light from the lamps 1 through the light transmitting windows 4a to 4f. Therefore, the substrate 6 to be processed can be evenly processed even though the width W_{W} of the light transmitting windows 4a to 4f in the above-mentioned moving direction is smaller than the length W_{B} of the substrate 6 in the moving direction.

### Second Embodiment

The first embodiment described above is an example of a substrate processing apparatus which swings a substrate 6 to be processed. The second embodiment is an example of a substrate processing apparatus which moves a large-sized substrate in one direction.

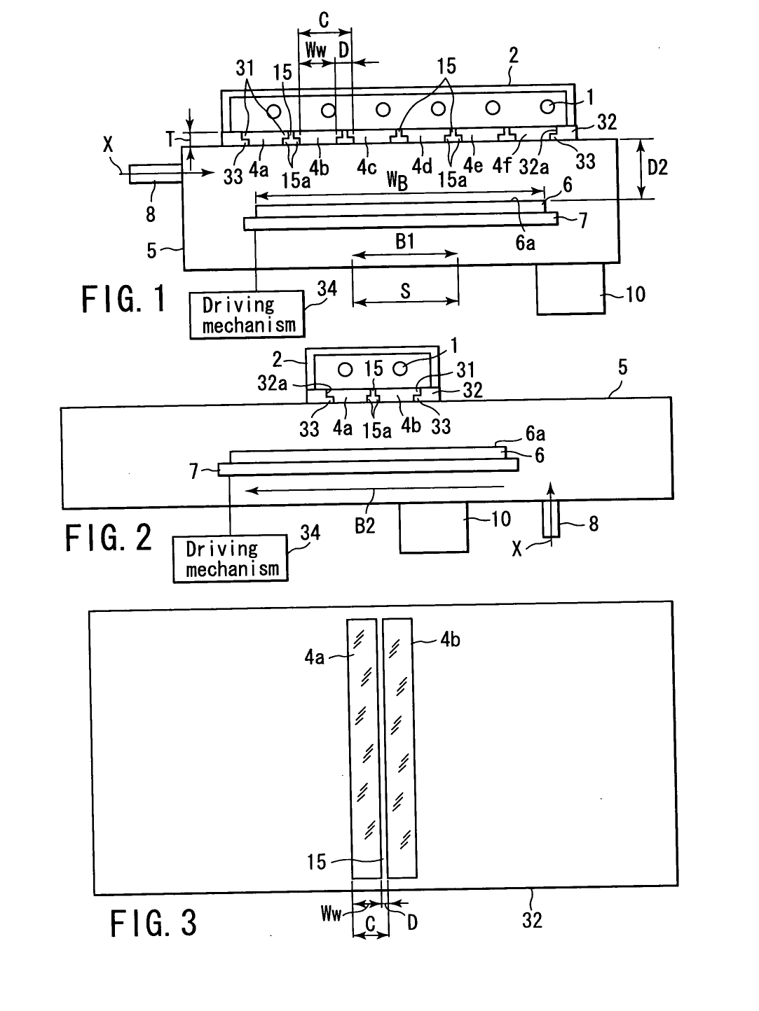

A plurality of (in this embodiment, two) xenon excimer lamps 1 as linear light sources are arranged parallel to each other so as to extend in the direction perpendicular to the paper of FIG. 2. Also, two thin and long light transmitting windows 4a and 4b (see FIG. 3,) oppose the juxtaposed linear light sources (xenon excimer lamps 1 not shown in FIG. 3) with a spacing between them. A driving mechanism 34 moves a substrate 6 to be processed in one direction, e.g., in a direction indicated by an arrow B2 in FIG. 2, with respect to the light transmitting windows 4a and 4b. To move the substrate 6 in one direction, the length of a reaction chamber 5 in this moving direction (indicated by the arrow B2 in FIG. 2) is made twice that of the substrate 6 in the moving direction B2 or more.

The substrate 6 to be processed is a 1,000×1,200-mm glass substrate. A width W_{W} of the light transmitting windows 4a and 4b is 90 mm, the thickness of the light transmitting windows 4a and 4b is 40 mm, and a width D of a beam 15 is 30 mm.

The substrate processing apparatus of the second embodiment can perform photo-oxidation on a surface 6a of the substrate 6 to be processed, while moving the substrate 6 in one direction (indicated by the arrow B2 in FIG. 2) below the light transmitting windows 4a and 4b.

Accordingly, even large substrates which are conventionally difficult to process can be processed. Note that the rate of photo-oxidation increases as the number of the light sources (xenon excimer lamps 1) increases. Therefore, the number of the light sources (xenon excimer lamps 1) is preferably determined so that a desired throughput is obtained.

### Third Embodiment

In the second embodiment described above, to move the substrate 6 to be processed in one direction, the length of the reaction chamber 5 in the moving direction B2 must be twice that of the substrate 6 in the moving direction B2 or more. The third embodiment improves the footprint by using an inline system.

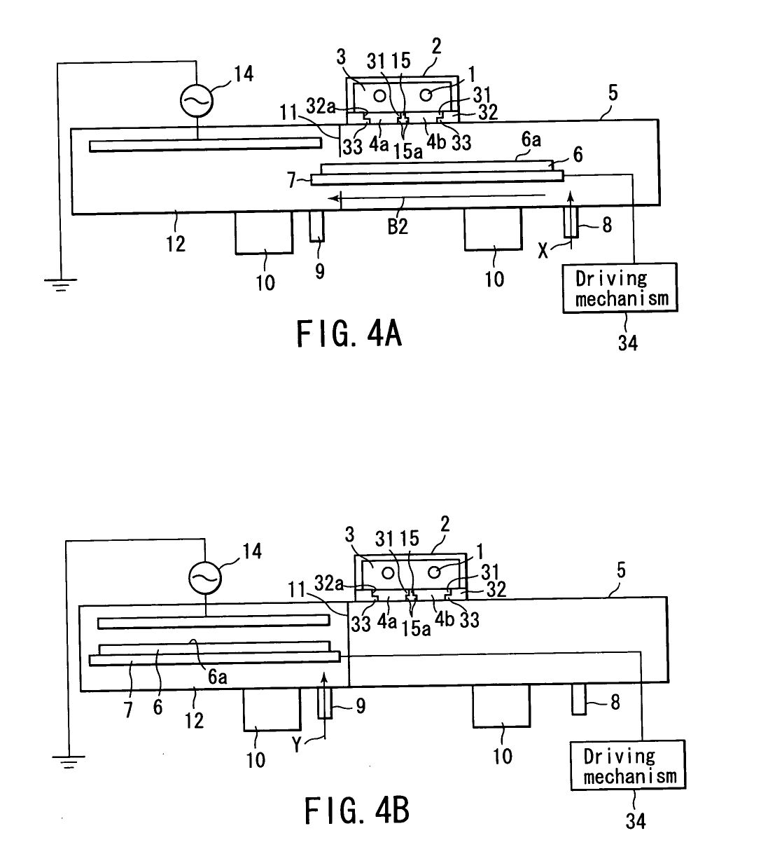

As shown in FIGS. 4A and 4B, a first reaction chamber 5 of a substrate processing apparatus of the third embodiment has a gave valve 11. A second reaction chamber 12 (as a sub-reaction chamber) different from the first reaction chamber 5 is positioned adjacent to the first reaction chamber 5 via the gate valve 11. The first reaction chamber 5 is a photo-oxidation chamber for performing photo-oxidation. The second reaction chamber 12 is a plasma CVD chamber for performing plasma CVD. The first reaction chamber 5 may also be placed adjacent to a plurality of reaction chambers via a plurality of gate valves 11. A driving mechanism 34 moves a substrate 6 to be processed in one direction from the first reaction chamber 5 to the second reaction chamber 12 over the gate valve 11. The moving direction of the substrate 6 is a direction indicated by an arrow B2 in FIG. 4A.

As described above, the substrate processing apparatus of the third embodiment uses an inline system in which the photo-oxidation chamber (reaction chamber 5) and plasma CVD chamber (reaction chamber 12) are connected via the gate valve 11. Accordingly, the footprint can be improved.

That is, to perform photo-oxidation on the substrate 6 to be processed, as shown in FIG. 4A, the gate valve 11 is opened to supply oxygen gas to both the photo-oxidation chamber (reaction chamber 5) and plasma CVD chamber (reaction chamber 12). As same as the second embodiment, photo-oxidation is performed while the substrate 6 is moved in the direction B2 over the gate valve 11. More specifically, photo-oxidation is performed as the substrate 6 is moved to the plasma CVD chamber, thereby forming a first insulating film.

When the substrate 6 is moved to the plasma CVD chamber 12, as shown in FIG. 4B, the gate valve 11 is closed. From a gas inlet 8 of the reaction chamber 12, a semiconductor gas 13 is supplied into the reaction chamber 12 as indicated by an arrow Y. After that, an RF voltage is applied from an RF power supply 14 to form a second insulating film (an SiO₂ film or another insulating film) by plasma CVD.

In this embodiment, as a reaction which occurs when the light emitted from the light source is irradiated into the reaction chamber through at least one light transmitting window, at least two of photo-oxidation, photo-CVD, photo-ashing, photo-cleaning, photo-etching, and photo-epitaxy can be continuously performed without breaking the vacuum.

### Fourth Embodiment

In each of the first, second, and third embodiments described above, a plurality of linear light sources (xenon excimer lamps 1) are arranged, and light transmitting windows are formed to oppose these light sources with a spacing between them. That is, the light transmitting windows are juxtaposed in a first direction, e.g., the moving direction.

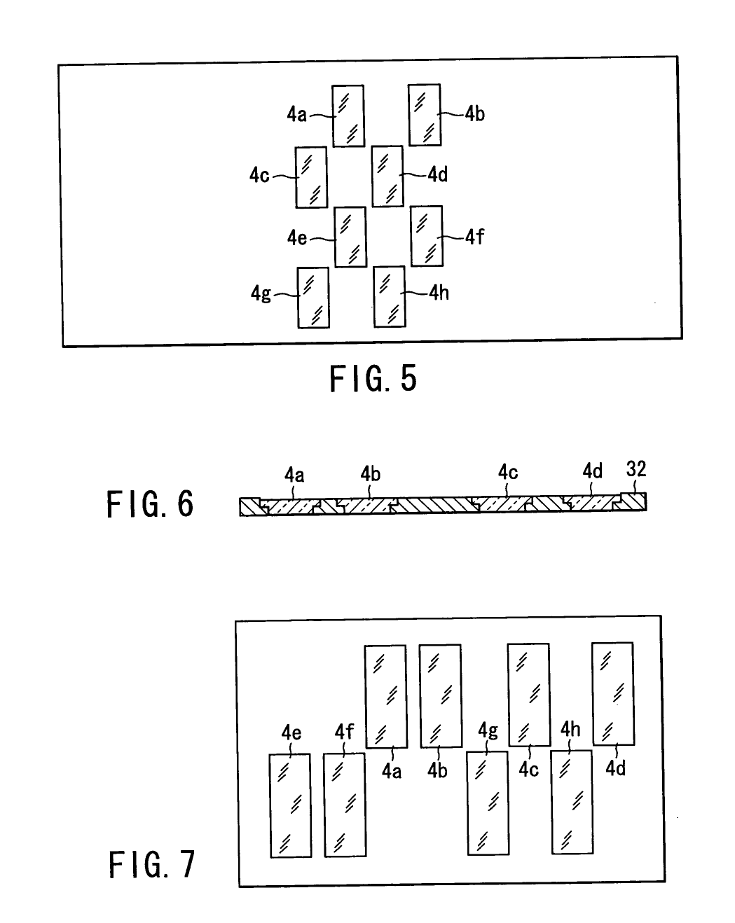

Light transmitting windows 4a to 4h, however, may also be juxtaposed in a first direction and a second direction different from the first direction. In the fourth embodiment, as shown in FIG. 5, the first direction is parallel to the moving direction described previously, and the second direction is parallel to linear light sources 1 and perpendicular to the moving direction.

To arrange the light transmitting windows 4a to 4h in the first and second directions different from each other, the light transmitting windows 4a to 4h can be arranged into a check pattern. With this arrangement, the thickness of the light transmitting windows 4a to 4h can be made further smaller than in the first to third embodiments. Also, short light sources corresponding to the size (length) of the light transmitting windows 4a to 4h can be used. Consequently, large substrates can be processed by short light sources.

### Fifth Embodiment

When a substrate processing apparatus has a plurality of light transmitting windows, the intervals between adjacent light transmitting windows in the moving direction of a substrate 6 to be processed need not always be constant repeating intervals. That is, the total light transmitting window width in the moving direction is essential. The fifth embodiment is an example in which the intervals between adjacent light transmitting windows in the moving direction are not uniform.

In the fifth embodiment, light transmitting windows 4a to 4d are juxtaposed in one direction, e.g., in a direction parallel to the moving direction described above. The intervals between the light transmitting windows 4a to 4d are not uniform. Light transmitting windows 4e to 4h are also juxtaposed in a direction parallel to the moving direction. The intervals between the light transmitting windows 4e to 4h are not uniform. That is, the light transmitting windows 4a to 4h are juxtaposed four by four in two lines.

To arrange the light transmitting windows 4a to 4h in a plurality of lines in the direction parallel to the moving direction, the total width of the light transmitting windows 4a to 4d in the moving direction is desirably equal to that of the light transmitting windows 4e to 4h in the same direction. This is to evenly process the surface of a substrate 6 to be processed. In the fifth embodiment, the light transmitting windows 4a to 4h are subjected to the same processing. That is, the total width of the light transmitting windows 4a to 4d and that of the light transmitting windows 4e to 4h in the moving direction are equal. Even with this arrangement, the same effect as in the first embodiment is obtained.

### Sixth Embodiment

The first embodiment is an example which uses a single-crystal silicon substrate as a substrate 6 to be processed. On the basis of the result of the first embodiment, the fabrication process of polysilicon thin-film transistors (poly-Si TFTs) for a liquid crystal display device formed on a glass substrate will be described below.

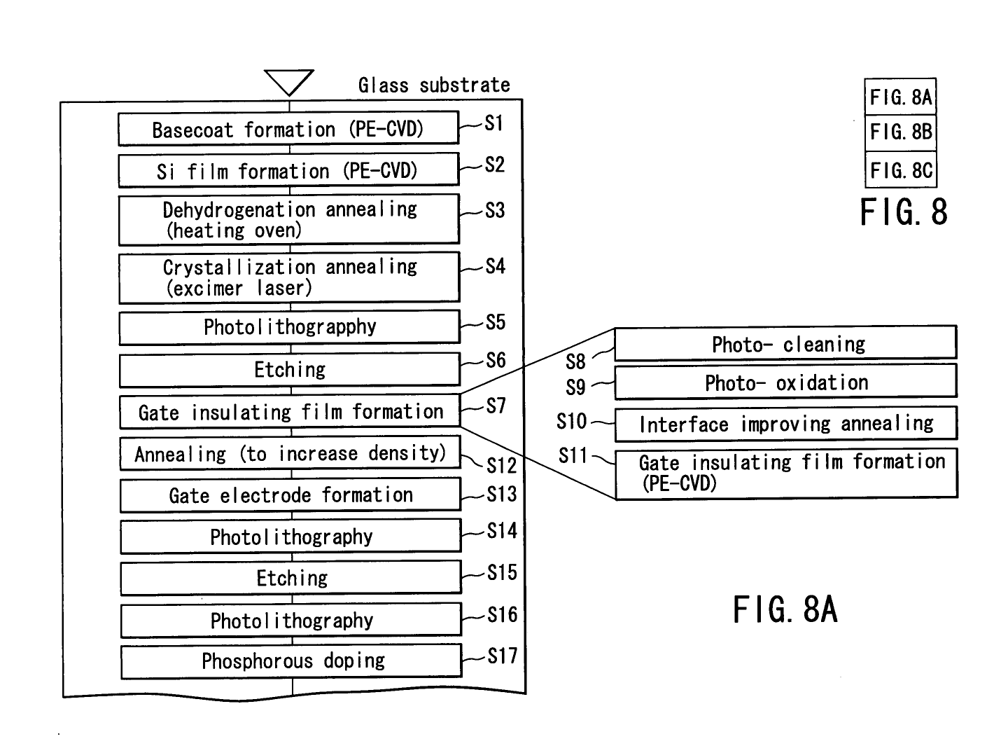

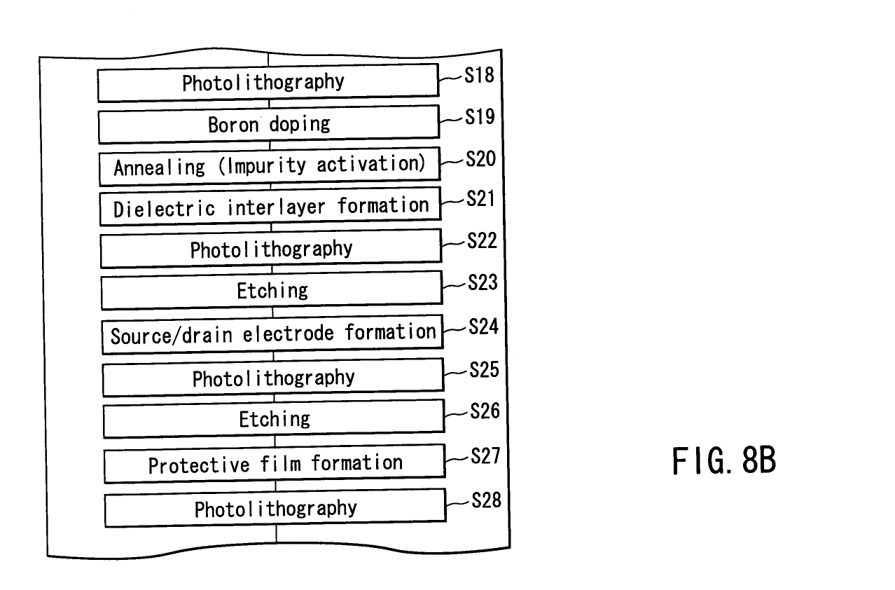

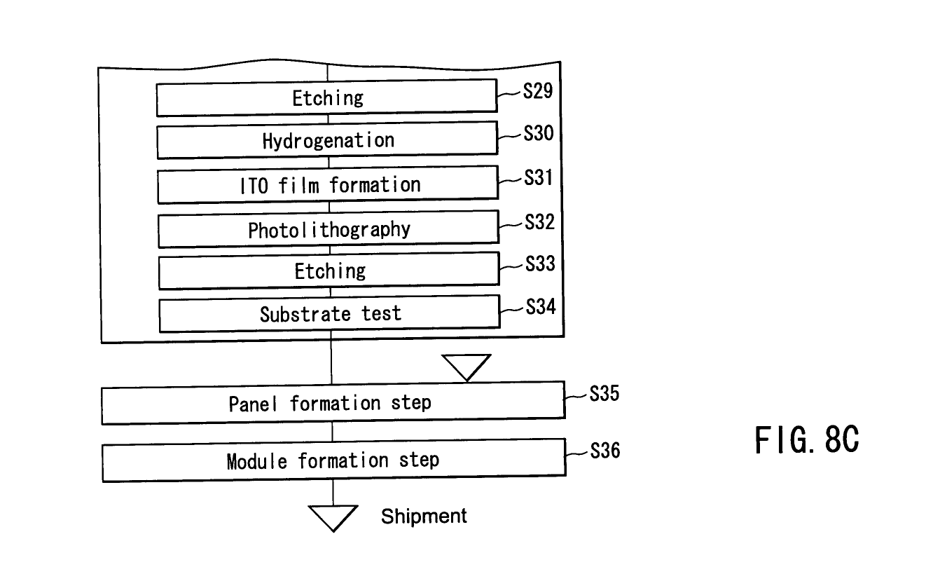

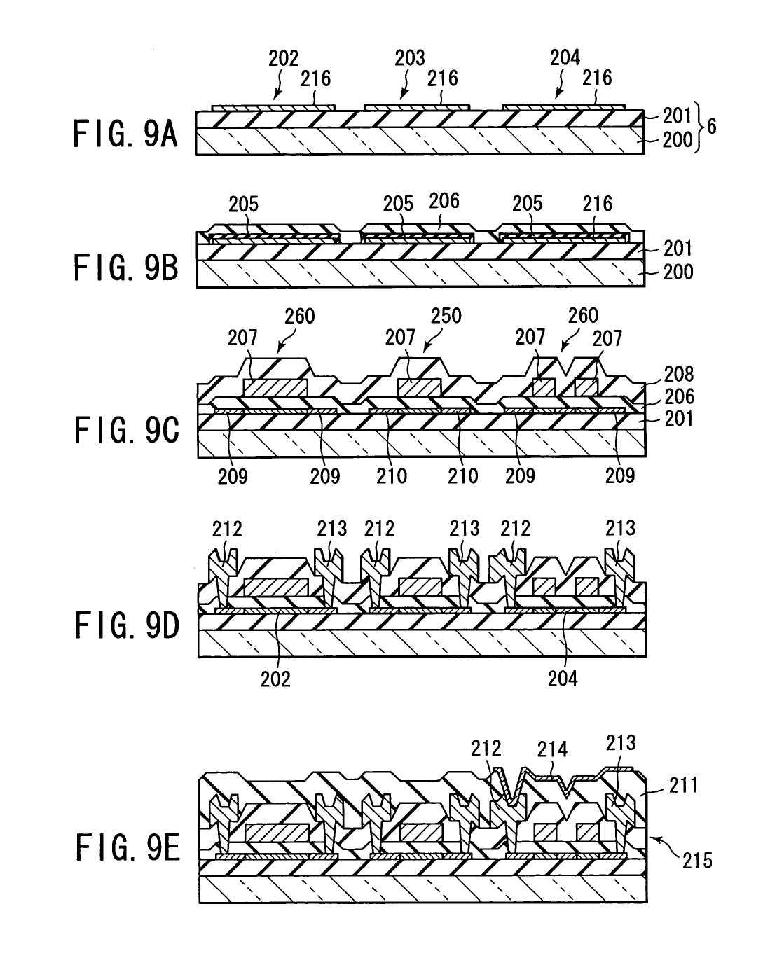

FIGS. 8A, 8B and 8C are a process flow charts when the present invention is applied to n- and p-channel polysilicon thin film transistors for forming a liquid crystal display device. FIGS. 9A to 9E are cross sectional views of the elements in individual processes.

As a glass substrate 200 (FIGS. 9A to 9E), a glass plate having dimensions of 320 mm×400 mm×1.1 mm is used.

As shown in FIG. 9A, on the cleaned glass substrate 200, a 200-nm thick silicon oxide film (SiO₂ film) is formed as a basecoat film 201 (FIG. 9A) by PE-CVD (Plasma Enhanced CVD) using TEOS gas (S1 in FIG. 8A).

After that, SiH₄ and H₂ gases are used to form a 50-nm thick amorphous silicon film by PE-CVD (S2 in FIG. 8A).

This amorphous silicon film contains hydrogen of 5 to 15 atom %. Therefore, if the film is directly irradiated with a laser, the hydrogen turns into a gas to cause abrupt volume expansion, thereby blowing off the film. To prevent this, the glass substrate 200 over which this amorphous silicon film is formed is held at 350° C. or more at which hydrogen bonds are broken for about 1 hr, thereby letting the hydrogen go (S3 in FIG. 8A) After that, pulse light (670 mJ/pulse) emitted from a xenon chloride (XeCl) excimer laser and having a wavelength of 308 nm is shaped into 0.8 mm×130 mm by an optical system. The amorphous silicon film on the glass substrate 200 is irradiated with the laser light at an intensity of 360 mJ/cm². The amorphous silicon melts by absorbing the laser light and turns into a liquid phase. This amorphous silicon is cooled and turn into a solid phase after that. In this way, polysilicon is obtained. The laser light is a 200 Hz pulse, and melting and solidifying are completed within the period of one pulse. Therefore, melting and solidifying are repeated for every pulse by laser irradiation. A large area can be crystallized by irradiating the glass substrate 200 with the laser while the glass substrate 200 is moved. To reduce the deviation of characteristics, irradiation is preferably so performed that the irradiation regions of the individual laser pulse beams overlap by 95% to 97.5% (S4 in FIG. 8A).

As shown in FIG. 9A, this polysilicon layer is patterned into island-like polysilicon layers 216 corresponding to a source, channel, and drain in a photolithography step (S5 in FIG. 8A) and an etching step (S6 in FIG. 8A), thereby forming an n-channel TFT region 202, p-channel TFT region 203, and pixel TFT region 204 (FIG. 9A shows the process up to this point).

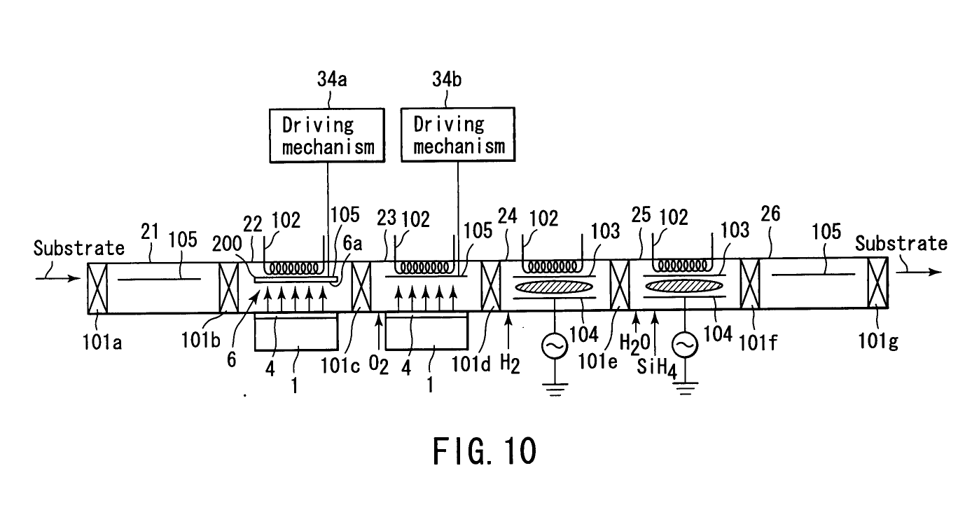

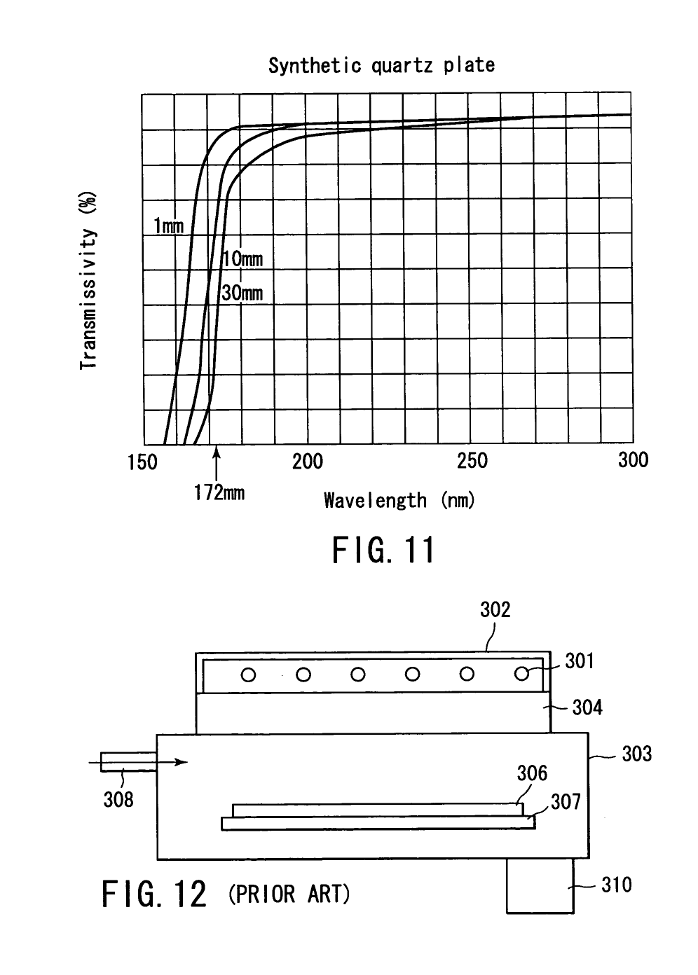

After that, an interface and insulating film which are most important in a poly-Si TFT are formed (S7 in FIG. 8A). That is, in the sixth embodiment, the glass substrate 200 processed to the state shown in FIG. 9A is equivalent to a substrate 6 to be processed. More specifically, the substrate 6 includes the glass substrate 200, the basecoat film 201 formed on the glass substrate 200, and the island-like polysilicon layers 216 formed on the basecoat. FIG. 10 is a side view schematically showing a thin film formation apparatus as a substrate processing apparatus which is a combination of a thin film formation apparatus which performs inline photo-oxidation, and a thin film formation apparatus which performs plasma CVD.

In FIG. 10, reference numeral 1 denotes xenon excimer lamps; 4, light transmitting windows made of synthetic quartz; 21, a loading chamber; 22, a photo-cleaning chamber; 23, a photo-oxidation chamber; 24, a hydrogen plasma chamber; 25, a film formation chamber; 26, an unloading chamber; 101a to 101g, gate valves; 102, heaters; 103, cathode electrodes; 104, anode electrodes; and 105, substrate holders. The substrate holders 105 in the photo-cleaning chamber 22 and photo-oxidation chamber 23 are swung by driving mechanisms 34a and 34b, respectively.

This substrate processing apparatus shown in FIG. 10 comprises the plurality of reaction chambers, the gate valves 101a to 101g as means for moving the substrate 6 between these reaction chambers without exposing it to the atmosphere. The reaction chambers include the photo-oxidation chamber 23 as a first reaction chamber for setting the glass substrate 200, and forming an insulating film by photo-oxidation, and the film formation chamber 25 as a second reaction chamber for setting the substrate 6, and forming a second insulating film by deposition on the first insulating film.

The gate valve 101a is opened to load the substrate 6 (FIG. 9A) having the island-like polysilicon layers 216 formed on the basecoat film 201 into the loading chamber 21. After that, the loading chamber 21 is evacuated, and the gate valve 101b is opened. The substrate 6 is moved to the photo-cleaning chamber 22, and the gate valve 101b is closed. After the substrate 6 is set on the substrate holder 105 at a temperature of 350° C., the silicon surface (the surfaces of the island-like silicon layers 216) is irradiated with light having a wavelength of 172 nm, which is emitted from the xenon excimer lamp 1 as a light source through the synthetic quartz light transmitting window 4, while the substrate 6 is swung. In this manner, the silicon surface is photo-cleaned to remove mainly resinous stains (S8 in FIG. 8A). The width of the light transmitting window 4 is 90 mm, the distance between the adjacent light transmitting windows 4 is 30 mm, the thickness of the light transmitting window 4 is 40 mm, and the stroke of the swing of the substrate 6 is 150 mm.

Although photo-cleaning can also be performed by using a low-pressure mercury lamp as a light source, the cleaning effect is higher when the xenon excimer lamp 1 is used. The light irradiation intensity after the light passes through the light transmitting window 4 is held at 45 mW/cm², and the distance from the light transmitting window 4 to the silicon surface is held at 25 mm.

After that, the gate valve 101c is opened to move the photo-cleaned substrate 6 to the photo-oxidation chamber 23 (the first reaction chamber for forming a first insulating film), and then the gate valve 101c is closed. The substrate 6 is set on the substrate holder 105 at a temperature of 350° C. Oxygen gas is supplied into the photo-oxidation chamber 23, and the internal pressure of the photo-oxidation chamber 23 is held at 70 Pa. In addition, while the substrate 6 is swung, the oxygen gas is irradiated with light from the xenon excimer lamp 1 which emits light having a wavelength of 172 nm, through the light transmitting window 4. Consequently, the oxygen gas is directly decomposed into highly active oxygen atoms. This active oxygen atoms oxidizes the island-like polysilicon layers 216 to form a photo-oxidized SiO₂ film which is a gate insulating film 205 (i.e., a first insulating film shown in FIG. 9B). The substrate processing apparatus of the sixth embodiment was able to form a gate insulating film 205 (first insulating film) having a film thickness of about 3 nm in 3 min (S9 in FIG. 8A). The width of the light transmitting window 4 is 90 mm, the distance between the light transmitting windows adjacent to each other in the moving direction is 30 mm, the thickness of the light transmitting window 4 is 40 mm, and the stroke of the swing of the substrate 6 is 150 mm.

After that, annealing is performed to improve the interface characteristics. The gate valve 101d is opened to move the substrate 6 processed as described above to the hydrogen plasma chamber 24, and the gate valve 101d is closed. While the substrate temperature, H₂ gas flow rate, and gas pressure are held at 350° C., 1,000 sccm, and 173 Pa (1.3 Torr), respectively, hydrogen plasma processing is performed for the photo-oxidized film for 3 min by setting the internal pressure of the hydrogen plasma chamber 24 at 80 Pa (0.6 Torr) and the RF power at 450 W (S10 in FIG. 8A). It is also possible to perform hydrogenation (S30 in FIG. 8C) instead of this hydrogen plasma processing.

Subsequently, the gate valve 101e is opened to move the substrate 6 to the film formation chamber 25 (the second reaction chamber for forming a second insulating film), and the gate valve 101e is closed. A gate insulating film 206 (second insulating film) which is an SiO₂ film is formed by plasma CVD by setting the substrate temperature at 350° C., the SiH₄ gas flow rate at 30 sccm, the N₂O gas flow rate at 6,000 sccm, the internal pressure of the film formation chamber 25 at 267 Pa (2 Torr), and the RF power at 450 W. The substrate processing apparatus of the sixth embodiment was able to form a 97 nm thick second gate insulating film 206 in 3 min (S11 in FIG. 8A).

After that, the gate valve 101f is opened to move the substrate 6 to the unloading chamber 26, and the gate valve 101f is closed. Then, the gate valve 101g is opened to unload the substrate 6 (FIG. 9B).

By the substrate processing apparatus shown in FIG. 10, the photo-cleaning step (S8 in FIG. 8A), the photo-oxidation step (S9 in FIG. 8A), the interface improving annealing step to improve interface (S10 in FIG. 8A), and the step of forming the first gate insulating film 205 by plasma CVD (S11 in FIG. 8A) can be continuously performed in a vacuum without lowering the productivity. Consequently, a good interface can be formed between the semiconductor (the island-like polysilicon layers 216) and the first gate insulating film 205, and a thick, highly practical insulating film can be rapidly formed.

After that, poly-Si TFTs are formed as follows.

The substrate 6 processed as above is annealed in nitrogen gas at a substrate temperature of 350° C. for 2 hrs, thereby increasing the density of the first gate insulating film 205 made of an SiO₂ film (S12 in FIG. 8A). This density increasing process raises the density of the SiO₂ film, thereby increasing the leakage current and breakdown voltage.

After a 100-nm thick Ti layer is formed as a barrier metal by sputtering, a 400 nm thick Al layer is similarly formed by sputtering (S13 in FIG. 8A). This Al layer is then patterned (S15 in FIG. 8A) by photolithography (S14 in FIG. 8A) to form gate electrodes 207 as shown in FIG. 9C.

After that, only a p-channel TFT 250 is covered with a photoresist (not shown) by photolithography (S16 in FIG. 8A). Ion doping is then performed by using the gate electrodes 207 as masks, thereby doping 6×10¹⁵/cm² of phosphorous at 80 keV into n⁺ source/drain contact portions 209 of n-channel TFTs 260 (S17 in FIG. 8A).

In a photolithography step, the n-channel TFTs 260 in the n-channel TFT region 202 and pixel TFT region 204 are covered with a photoresist (S18 in FIG. 8B). Ion doping is then performed by using the gate electrodes 207 as masks, thereby doping 1×10¹⁶/cm² of boron at 60 keV into p⁺ source/drain contact portions 210 of the p-channel TFT 250 (FIG. 9C) in the p-channel region 203 (FIG. 9A) (Sl9 in FIG. 8B).

The substrate 6 processed as above is annealed at a substrate temperature of 350° C. for 2 hrs to activate the ion-doped phosphorous and boron (S20 in FIG. 8B). Plasma CVD using TEOS gas is then performed to form a dielectric interlayer 208 made of SiO₂ (S21 in FIG. 8B) (FIG. 9C).

Subsequently, in a photolithography step (S22 in FIG. 8B) and an etching step (S23 in FIG. 8B), contact holes reaching the n⁺ source/drain contact portions 209 and p⁺ source/drain contact portions 210 are formed by patterning as shown in FIG. 9D. Then, a 100-nm thick Ti layer is formed as a barrier metal (not shown) by sputtering, a 400-nm thick Al layer is formed by sputtering (S24 in FIG. 8B). In addition, source electrodes 213 and drain electrodes 212 are formed by patterning (FIG. 9D) in a photolithography step (S25 in FIG. 8B) and an etching step (S26 in FIG. 8B).

Furthermore, as shown in FIG. 9E, a 300 nm thick passivation film 211 made of SiO₂ is formed by plasma CVD (S27 in FIG. 8B). To bare the drain region 212 of the n-channel TFT 260 (FIG. 9C) in the pixel TFT region 204 (FIG. 9A), a contact hole for connecting to an ITO pixel electrode 214 (to be described later) is formed by patterning in a photolithography step (S28 in FIG. 8B) and an etching step (S29 in FIG. 8C).

After that, a gas mixture of nitrogen gas flow rate:hydrogen gas flow rate=97:3 is supplied into a hydrogen annealing oven at a substantially atmospheric pressure, thereby annealing the substrate 6 at a substrate temperature of 400° C. for 80 min. If the hydrogen plasma processing described previously is omitted, this process must be performed for 1 hr under the same conditions as above.

The substrate 6 is then moved to another reaction chamber to form a 150 nm thick ITO film (S31 in FIG. 8C). A pixel electrode 214 is formed by patterning this ITO film in a photolithography step (S32 in FIG. 8C) and an etching step (S33 in FIG. 8C). In this way, a TFT substrate 215 is completed (FIG. 9E). After that, a substrate test is performed (S34 in FIG. 8C).

The TFT substrate 215 and a glass substrate (not shown) having a color filter (not shown) are coated with polyimide, rubbed, and adhered to each other. The adhered substrates are divided into panels.

These panels are placed in a vacuum chamber, injection ports of the panels are dipped in a liquid crystal in a dish, and air is supplied into the chamber to inject the liquid crystal into the panels by the air pressure. The injection ports are then encapsulated with a resin to complete liquid crystal panels (S35 in FIG. 8C).

After that, a polarizer is adhered, and a peripheral circuit, backlight, bezel, and the like are assembled to complete a liquid crystal module (S36 in FIG. 8C).

This liquid crystal module can be used in a personal computer, monitor, television set, portable terminal, or the like.

The threshold voltage of a conventional TFT in which an SiO₂ film was formed by plasma CVD without forming any photo-oxidized film was 1.9±0.8 V. In the sixth embodiment, however, the characteristics of the interface between the silicon oxide film and polysilicon (island-like polysilicon layers 216) and the insulating film bulk characteristics improved. Consequently, the threshold voltage of the TFT improved to 1.5±0.6 V. Since the deviations in threshold voltage were reduced, the production yield greatly improved. In addition, the power consumption was reduced by 10% because the driving voltage was lowered. Note that a good SiO₂/Si (silicon oxide film and polysilicon) interface was formed by photo-cleaning and photo-oxidation, so no contamination by Na ion and the like occurred. This reduced the changes in threshold voltage and improved the reliability.

The present invention has been described in detail above on the basis of the first to sixth embodiments. However, the present invention is not limited to the above embodiments and can of course be modified without departing from the spirit and scope of the invention.

For example, the present invention is applicable to the single-crystal silicon substrate surface in the first embodiment, the polysilicon layer and the like on the glass substrate in each of the second to sixth embodiments, and a single-crystal silicon layer, polysilicon layer, and the like on various substrates such as a plastic substrate.

Also, the present invention can be applied to a wide variation of semiconductor devices such as a single-crystal silicon MOS transistor, as well as a thin-film transistor. Furthermore, the present invention is applicable to a substrate processing apparatus which has a high photo-oxidation rate in photo-oxidation capable of forming a good semiconductor-insulating film interface, and which can process large-sized substrates.

Although photo-oxidation is explained in the first to fifth embodiments described above, the present invention is also applicable to photo-CVD, photo-ashing, photo-cleaning, photo-etching, photo-epitaxy, and the like. In addition, the present invention can use two or more of these photo-reactions without breaking a vacuum.

In photo-oxidation, a low-pressure mercury lamp can be used as a light source as described previously. It is also possible to use a rare gas excimer lamp. A xenon excimer lamp, krypton excimer lamp, and argon excimer lamp can emit light having wavelengths of 172, 146, and 126 nm, respectively. In particular, a xenon excimer lamp which emits light having a wavelength of 172 nm is suited to producing active oxygen atoms from oxygen gas.

Furthermore, the substrate processing method of the present invention can form a semiconductor film on the substrate 6 to be processed by forming, in the reaction chamber 5, an ambient of a gas of a compound having an atom which belongs to group 14 (C, Si, Ge, Sn, and Pb) of the periodic table or a gas mixture containing this gas, an ambient of a gas mixture containing a gas of a compound having an atom which belongs to group 13 (B, Al, Ga, In, and Tl) of the periodic table and a gas of a compound having an atom which belongs to group 15 (N, P, As, Sb, and Bi) of the periodic table, an ambient of a gas mixture containing a gas of a compound having an atom which belongs to group 12 (Zn, Cd, and Hg) of the periodic table and a gas of a compound having an atom which belongs to group 16 (O, S, Se, Te, and Po) of the periodic table, or an ambient of a gas containing at least a silicon compound gas.

Examples of the gas of a compound having an atom which belongs to group 14 of the periodic table are a silicon compound gas and GeH₄. Examples of the silicon compound gas are silane gases (SiH₄, Si₂H₆, and Si₃H₈), SiCl₄, SiH₂F₂, SiH₂Cl₂, Si(CH₃)₂H₂, and TEOS (TetraEthylorthoSilicate, Si(OC₂H₅)₄). One of these gases or a gas mixture of two or more of these gases is supplied into the reaction chamber 5, and photo-processing is performed in the same manner as in the first to sixth embodiments. In this way, Si compound films (e.g., an Si film, SiC film, SiGe film, SiO film, and SiO₂ film) can be formed. Note that the relationship between a gas supplied to the reaction chamber 5 and a film to be formed is already known.

An example of the gas mixture containing a gas of a compound having an atom which belongs to group 13 of the periodic table and a gas of a compound having an atom which belongs to group 15 of the periodic table is a gas mixture of Ga(C₂H₅)₃ and AsH₃. A GaAs film can be formed by supplying this gas mixture to the reaction chamber 5, and performing photo-processing in the same manner as in the first to sixth embodiments.

An example of the gas mixture containing a gas of a compound having an atom which belongs to group 12 of the periodic table and a gas of a compound having an atom which belongs to group 16 of the periodic table is a gas mixture of dimethylcadmium (DMCd) and diethyltellurium (DETe). A CdTe film can be formed by supplying this gas mixture to the reaction chamber 5, and performing photo-processing in the same manner as in the first to sixth embodiments.

Additional advantages and modifications will readily occur to those skilled in the art. Therefore, the invention in its broader aspects is not limited to the specific details and representative embodiments shown and described herein. Accordingly, various modifications may be made without departing from the spirit and scope of the general inventive concept as defined by the appended claims and their equivalents.

What is claimed is:

1. A substrate processing apparatus which comprises a light source, a light transmitting window which transmits light from the light source, and a reaction chamber capable of being evacuated, and in which a substrate to be processed is placed in the evacuated reaction chamber so as to oppose the light transmitting window with a spacing therebetween, and at least a surface to be processed of the substrate, which opposes the light transmitting window is processed by using a reaction which occurs when the light from the light source is irradiated into the reaction chamber through the light transmitting window,
comprising a driving mechanism which moves the substrate relative to the light transmitting window in a direction parallel to the surface to be processed, wherein a width of the light transmitting window in the direction in which the substrate moves relative to the light transmitting window is smaller than a length of the substrate in the moving direction.

2. A substrate processing apparatus which comprises a light source, a plurality of light transmitting windows which transmit light from the light source, and a reaction chamber capable of being evacuated, and in which a substrate to be processed is placed in the evacuated reaction chamber so as to oppose the light transmitting windows with a spacing therebetween, and at least a surface to be processed of the substrate, which opposes the light transmitting windows is processed by using a reaction which occurs when the light from the light source is irradiated into the reaction chamber through the light transmitting windows,
comprising a driving mechanism which moves the substrate relative to the light transmitting windows in a direction parallel to the surface to be processed, wherein a width of each of the light transmitting windows in the direction in which the substrate moves relative to the light transmitting windows is smaller than a length of the substrate in the moving direction.

3. An apparatus according to claim 2, wherein the light transmitting windows are juxtaposed in a first direction.

4. An apparatus according to claim 2, wherein the light transmitting windows are juxtaposed in a first direction and a second direction different from the first direction.

5. An apparatus according to claim 4, wherein the light transmitting windows are arranged into a check pattern.

6. An apparatus according to claim 1, wherein the driving mechanism swings the substrate with respect to the light transmitting windows.

7. An apparatus according to claim 2, wherein the driving mechanism swings the substrate with respect to the light transmitting windows.

8. An apparatus according to claim 7, wherein the light transmitting windows are juxtaposed in the swinging direction such that widths of the light transmitting windows in the swinging direction are constant, and intervals between adjacent light transmitting windows in the swinging direction are constant, and a stroke of the swing by the driving mechanism is larger than a repeating interval which is a sum of the width in the swinging direction of the light transmitting window and a width in the swinging direction of a beam formed between adjacent light transmitting windows.

9. An apparatus according to claim 2, wherein the light transmitting windows are juxtaposed in the moving direction such that intervals between adjacent light transmitting windows in the moving direction are not uniform.

10. An apparatus according to claim 1 or 2, wherein the driving mechanism moves the substrate in one direction with respect to the light transmitting windows.

11. An apparatus according to claim 10, wherein a length of the reaction chamber in the moving direction is more than twice a length of the substrate in the moving direction.

12. An apparatus according to claim 1 or 2, wherein the reaction chamber has a gate valve, at least one sub-reaction chamber different from the reaction chamber is placed adjacent to the reaction chamber via the gate valve, and the driving mechanism moves the substrate in one way from the reaction chamber to the sub-reaction chamber over the gate valve.

13. An apparatus according to claim 1 or 2, wherein the light source is a low-pressure mercury lamp.

14. An apparatus according to claim 1 or 2, wherein the light source is a rare gas excimer lamp.

15. An apparatus according to claim 14, wherein the light source is a xenon excimer lamp.

16. A substrate processing method comprising steps of:
placing a substrate to be processed in an evacuated reaction chamber of a substrate processing apparatus comprising a light source, at least one light transmitting window which transmits light from the light source, and the reaction chamber capable of being evacuated, such that the substrate opposes the light transmitting window with a spacing therebetween;
irradiating the reaction chamber by the light from the light source through the light transmitting window, while moving the substrate relative to the light transmitting window such that the substrate is parallel to the light transmitting window; and
processing at least a surface to be processed of the substrate, which opposes the light transmitting window, by a reaction which occurs when light from the light source is irradiated into the reaction chamber.

17. A method according to claim 16, which further comprises steps of:
preparing a substrate to be processed having a surface to be processed which is at least partially made of a semiconductor; and
forming an ambient containing at least oxygen gas in the reaction chamber,
wherein the step of processing at least the surface to be processed of the substrate comprises a step of oxidizing the surface to be processed by using active oxygen atoms formed by the reaction which occurs when light from the light source is irradiated into the reaction chamber, thereby forming an insulating film on the substrate.

18. A method according to claim 16, which further comprises a step of forming, in the reaction chamber, an ambient of a gas of a compound having an atom which belongs to group 14 of the periodic table or a gas mixture containing the gas, an ambient of a gas mixture containing a gas of a compound having an atom which belongs to group 13 of the periodic table and a gas of a compound having an atom which belongs to group 15 of the periodic table, an ambient of a gas mixture containing a gas of a compound having an atom which belongs to group 12 of the periodic table and a gas of a compound having an atom which belongs to group 16 of the periodic table, or an ambient of a gas containing at least a silicon compound gas,
wherein the step of processing at least the surface to be processed of the substrate comprises a step of forming a semiconductor film on the substrate by the reaction which occurs when light from the light source is irradiated into the reaction chamber.

19. A method according to claim 16, wherein photo-oxidation, photo-CVD, photo-ashing, photo-cleaning, photo-etching, or photo-epitaxy is used as the reaction which occurs when the interior of the reaction chamber is irradiated with the light from the light source through at least one light transmitting window.

20. A method according to any one of claims 16 to 18, wherein at least two of photo-oxidation, photo-CVD, photo-ashing, photo-cleaning, photo-etching, and photo-epitaxy are continuously performed without breaking a vacuum.
